(19) United States
(12) Patent Application Publication
(10) Pub. No.: US 2011/0129827 A1
(43) Pub. Date: Jun. 2, 2011

(54) METHODS FOR TRANSCRIPT ANALYSIS

(75) Inventors: Marie Causey, Belmont, MA (US); Tal Raz, Brookline, MA (US); Doron Lipson, Chestnut Hill, MA (US)

(73) Assignee: Helicos Biosciences Corporation, Cambridge, MA (US)

(21) Appl. No.: 12/811,579

(22) PCT Filed: Apr. 3, 2009
(86) PCT No.: PCT/US09/39477

§ 371 Date: Oct. 25, 2010

Related U.S. Application Data
(60) Provisional application No. 61/042,460, filed on Apr. 4, 2008.
(60) Provisional application No. 61/044,310, filed on Apr. 11, 2008.

(51) Int. Cl. C12Q 1/68

(52) U.S. Cl. 435/6.11

(57) ABSTRACT

The invention takes a unique approach to transcript analysis that provides a novel DGE technology based on single-molecule sequencing. More particularly, the invention relates to methods and compositions for analyzing and identifying genes and gene expression and transcript profiles using a DGE-based technology and single molecule sequencing that does not require amplification or fragmentation.

### RELATED APPLICATIONS

The invention is a national phase application and claims the benefit of PCT/US2009/039477, filed Apr. 3, 2009, which is related to and claims the benefit of U.S. provisional patent application Ser. Nos. 61/042,460, filed Apr. 4, 2008, and 61/044,310, filed Apr. 11, 2008 with the U.S. Patent and Trademark Office, each of which is incorporated herein by reference in its entirety for all purposes.

### TECHNICAL FIELD OF THE INVENTION

The invention generally relates to methods for transcript analysis. More particularly, the invention relates to methods and compositions for analyzing and identifying genes and gene expression and transcript profiles.

### BACKGROUND OF THE INVENTION

Gene expression analysis is an important technique for identifying genes, gene expression patterns that are important in disease and therapeutics, and for elucidating gene regulation and other regulatory mechanisms. For example, the availability of RNA profiling technologies has increased knowledge of the involvement of genes in disease as well as the identification of small molecule therapeutics. Identification and quantification of differentially expressed genes in cancer and other diseases are useful in diagnosis, prognosis, and treatment of those conditions. Quantitative gene expression enables precise identification, monitoring, and possible treatment at the molecular level.

Analysis of gene expression has been a primary tool in the study of cellular mechanisms. Large-scale sequencing of cDNA clones and comparisons of transcript abundance between samples have provided invaluable insight into the gene content of a wide range of organisms as well as tissue-specific and developmental patterns of expression. More recently, microarray expression profiling has provided information on gene expression. (Lockhart, et al. Nature 405, 827-836 (2000); Churchill Nat Genet 32 Suppl, 490-495 (2002).) There are, however, several significant limitations to hybridization-based technologies. First, the ability to accurately measure low-abundance transcripts is limited. Second, novel transcript discovery is not possible. Third, direct comparison of transcripts within an individual sample is inaccurate because hybridization kinetics for individual mRNAs are sequence-dependent, necessitating ratiometric comparison between paired samples.

Several Digital Gene Expression (DGE) technologies, such as Serial Analysis of Gene Expression (SAGE) and Massively Parallel Signature Sequencing (MPSS) and similar technologies have been developed in an attempt to efficiently sequence and count large numbers of transcripts. (Velculescu, et al. Science 270, 484-487 (1995); Brenner, et al. Nat Biotechnol 18, 630-634 (2000); Saha, et al. Nat Biotechnol 20, 508-512 (2002); Shiraki, et al. Proc Natl Acad Sci USA 100, 15776-15781 (2003); Hashimoto, et al. Nat Biotechnol 22, 1146-1149 (2004); Kim, et al. Science 316, 1481-1484 (2007).) Those techniques are based upon the assumption that a very short signature sequence is sufficient to identify a gene.

In general, DGE consists of high-throughput sequencing of short cDNA fragments (tags) that are matched to a reference transcriptome to identify the corresponding gene. Individual transcript abundances are then inferred from the relative tag counts for each gene in a “digital” manner, in contrast to the “analog” nature of microarray intensity-based quantification. To date, most SAGE-like strategies rely on cDNA restriction digestion, adaptor ligation and additional sample manipulation steps. This extensive sample manipulation, as well as the fact that tags are generated only from one or few limited sequence contexts per transcript, is likely to be the source of a number of transcript quantification biases that were recently described. (Chen, et al. BMC Genomics 7, 77 (2006); Siddiqui, et al. Nucleic Acids Res 34, e83 (2006); Gilchrist, et al. BMC Bioinformatics 8, 403 (2007); Hene, et al. BMC Genomics 8, 333 (2007); So, et al. Biotechnol Bioeng 94, 54-65 (2006).)

Recent studies have demonstrated that high-throughput short-read sequencing platforms can be used to generate high-resolution maps of complete transcriptomes by sequencing a significant fraction of the transcriptome at sufficient depth. (Nagalakshmi, et al. Science 320, 1344-1349 (2008); Mortazavi, et al. Nat Methods 5, 621-628 (2008); Cloonan, et al. Nat Methods 5, 613-619 (2008).) Since a different number of reads is generated from each mRNA molecule, extraction of quantitative measurements from full transcriptome sequencing data relies on an assessment of coverage depth for each transcript. While this approach indeed yields informative transcript quantification, it is costly in terms of the sheer number of reads that are required to completely cover an entire transcriptome (several tens of millions of reads per sample), limiting scalability.

Current transcript profiling methods involve cumbersome sample preparation and are susceptible to sample bias. For example, most sample preparation methods introduce amplification and/or capture bias that will reduce the accuracy of the resulting sequence analysis. Moreover, traditional transcript profiling requires numerous processing steps, each of which may be a potential point at which bias is introduced.

### SUMMARY OF THE INVENTION

The present invention takes a unique approach to transcript analysis that provides a novel DGE technology based on single-molecule sequencing. (Harris, et al. Science 320, 106-109 (2008).) Since no PCR amplification is employed, sample preparation does not necessitate the addition of adaptors to the cDNA, thus enabling a simple procedure that is free of restriction digestion, ligation or amplification steps. This methodology generates strand specific, accurate transcript counts covering the complete cellular dynamic range. Single-molecule sequencing DGE (smsDGE) is optimized for mRNA quantification rather than full transcriptome sequencing. The effectiveness of counting by smsDGE is driven by the fact that only a single read is generated from each cDNA molecule, thereby maintaining a faithful representation of transcript distribution in the data and alleviating the burden of covering the entire transcriptome sequence. smsDGE generates sequence reads from the 3′ ends of first-strand cDNA molecules and does not require the cDNA to be full length. Consequently, it works equally well with short cDNAs generated by incomplete reverse transcription or partial mRNA degradation.

smsDGE involves the hybridization of poly-A tailed first strand cDNA molecules to oligonucleotide primers attached to the surface of a flow cell. The cDNA is then sequenced by single-molecule imaging of the stepwise addition of fluorescently-labeled nucleotides onto the surface. The sequencing reaction does not require any amplification steps, allowing strands to be densely packed onto the flow cell surface resulting in extremely high throughput (tens of millions of strands per channel).

As discussed herein, smsDGE has been successfully applied to the Saccharomyces cerevisiae DBY746 transcriptome, providing accurate abundance levels of all transcripts in a single channel of a HELICOS sequencer, a description of which is found at the HELICOS website.

Therefore, the present invention provides methods for gene expression analysis. Methods of the invention reduce sample bias and provide improved transcript counting and information content. Methods of the invention provide the ability to count individual RNA (cDNA) molecules, which leads to the ability to detect rare transcripts, to identify mutations (e.g., single nucleotide polymorphisms or SNPs), splice variants, and new genes/transcripts. The digital nature of methods of the invention enables comparison of expression levels from different genes within the same sample as well as comparisons of the same or different genes across different samples.

In one aspect, the invention generally relates to a method for analyzing RNA transcripts. The method includes sequencing a first strand cDNA via single-molecule sequencing thereby obtaining transcript information, wherein the method does not comprise a step of RNA or cDNA amplification. In certain embodiments, the method does not comprise RNA or cDNA fragmentation. In certain detailed embodiments, the method includes the steps of: copying a RNA to form a cDNA; polyadenylating the cDNA; hybridizing the polyadenylated cDNA to a primer bound to a surface; and conducting single-molecule sequencing of the cDNA.

In one embodiment, the invention features amplification-free 5′ mRNA sample preparation. There are several variations of sample preparation according to the invention. In one variation a Messenger RNA (mRNA) is primed with poly-deoxyribonucleoside thymidine (poly dT or oligo dT) using reverse transcriptase. After priming, a cDNA copy is made. The mRNA portion is removed and the remaining cDNA copy is polyadenylated and then is ready for sequencing as described herein. A schematic showing this variation of amplification-free sample preparation is show in FIG. 1.

In another variation, random oligomer priming is used instead of oligo dT priming. Thus, random primers are placed along all or a portion of the mRNA followed by cDNA synthesis as described above. The result is a series of cDNA copies that represent most or all of the mRNA template.

For transcript counting, the use of oligo dT priming as described above is preferred. For total transcriptome sequencing, in which coverage is more important that counting, either method can be used. However, random oligomer priming, since it primes first strand cDNA synthesis at various sites along the mRNA, increases the likelihood that the entire mRNA will be represented in the resulting cDNA mixture.

As described herein, amplification-free sample preparation avoids errors introduced during amplification, does not require fragmentation of cDNA, and does not suffer from bias introduced through the amplification process. This results in accurate counting and/or representation of mRNA present in a biological sample.

In preferred embodiments, methods of cDNA synthesis as described above may include the addition of a standard dNTP mixture comprising dATP, dCTP, dGTP and dTTP; or may include varying amounts of dUTP. The incorporation of small amounts of dUTP is useful to generate strands that have random dU incorporations in the place of dT. In that embodiment, after cDNA synthesis, the cDNA is treated with, for example, USER enzyme (New England Biolabs), which is a mixture of uracil DNA glycosylase and DNA glycosylase-lyase Endonuclease VIII (New England Biolabs) to cleave the first strand cDNA at all dU incorporations, creating a randomly fragmented cDNA sample that is representative of all or a portion of the mRNA transcript. By varying the amount of dU in the dNTPs mixture, larger or smaller fragments can be generated by USER digestion. The digestion via USER is complete and simple to control via dUTP concentration. In other embodiments, cDNA can also be fragmented with DNase I or with other endonucleases if dU is not incorporated. In general, fragmentation is useful to obtain sequenceable subsequences from the entire transcript and therefore obtain better sequencing representation of the transcriptome. Finally, following first strand cDNA synthesis, USER enzyme may be used to remove a dU primer if it was used.

In another embodiment, methods of cDNA synthesis as described above may include the addition of a standard dNTP mixture comprising dATP, dCTP, dGTP and dTTP that also includes varying amounts of ribonucleoside triphosphates (rNTPs). After synthesis, the RNA/DNA hybrid may be treated with a mixture of RNase H and Ribonuclease HII (RNase HII, New England Biolabs). RNase H degrades the RNA strand. RNase HII is an endoribonuclease that preferentially nicks 5′ to a ribonucleotide within the context of a DNA duplex. The enzyme leaves 5′ phosphate and 3′ hydroxyl ends. This results in the fragmentation of the cDNA and will increase the number of 5′ ends that can be tailed in the following step. By varying the amount of rNTPs in the dNTP mixture, larger or smaller fragments can be generated by RNase II digestion. To reiterate, fragmentation is useful to obtain subsequences from the entire transcript and therefore obtain better sequencing representation of the transcriptome.

In another embodiment, methods of cDNA synthesis as described above may include the addition of a standard dNTP mixture comprising dATP, dCTP, dGTP and dTTP that also includes varying amounts of terminating nucleotides (e.g. dideoxyribonucleotide triphosphates (ddNTPs), acyclonucleotides (acyNTPs), reversible terminators, or any modified nucleotides that restrict chain elongation during DNA polymerization). Random incorporation of chain terminating nucleotides will result in a greater range of 5′ termini of cDNAs, which after tailing and sequencing, will provide greater sampling of the mRNA sequence space by the short read sequencing technology described herein. In principle, any nucleotide that disrupts chain extension may be used for this embodiment of the Invention.

After cDNA synthesis, a polyA tail is generated on the free 3′ OH of all cDNA fragments. The tail is enzymatically generated using terminal deoxynucleotide transferase (TdT) and dATP. Typically, a polyA tail comprising about 50 to about 70 dA nucleotides is used. The polyA tail facilitates hybridization of the cDNA to polyT primer molecules attached to a surface for sequencing as described below. In principle, polynucleotide tailing can be carried out with a variety of dNTPs (or heterogeneous combinations) including but not limited to dATP. However, dATP is preferred because TdT adds dATP with predictable kinetics useful to synthesize a 50-70 nucleotide tail.

In one embodiment, in which accurate counting of transcripts is desired, cDNA is prepared such that only one cDNA is produced per mRNA molecule obtained from the starting sample. In a preferred embodiment, priming with oligo dT or dU is used, producing cDNA without fragmentation prior to dA tailing. The cDNA produced pursuant to this embodiment results in sequencing reads that are generated from the 5′-most region of the cDNAs. For short mRNAs, this may also correspond to the 5′ end of the nascent mRNA. For long mRNAs, a full-length cDNA copy is not often generated due to limitations in the ability of the reverse transcriptase enzyme to synthesize lengthy cDNA molecules. In such cases, a partial cDNA is generated that can then be polyA tailed and sequenced.

In another embodiment, in which full transcriptome sequencing is desired, cDNA is prepared by any priming method (see above) and subsequently fragmented as desired. Fragmentation results in the substantial loss of single cDNA/mRNA representation, but greatly increases the portion of the mRNA that can be sampled by short read sequencing technology as described herein.

Samples for use in the invention may be obtained from whole organisms, cell lines, tissue, blood, bodily fluids, or any other mRNA source. Methods of the invention are especially useful in combination with single molecule sequencing techniques, such as are described in co-owned U.S. Pat. No. 7,282,337, and co-owned U.S. patent application Ser. No. 11/496,275 (filed Jul. 31, 2006, Publication No. 2008-0026381 A1), each of which is incorporated by reference herein. Single molecule sequencing, which comprises sequencing individual strands of DNA or RNA on a surface such that each strand is individually optically resolvable, provides inexpensive, high-throughput, and accurate analysis of nucleic acids and preserves the digital nature of the sample.

Once cDNA is prepared, in a preferred embodiment, sequencing is conducted on a surface onto which are attached primers for sequencing-by-synthesis. In embodiments in which cDNA is polyA tailed, primers are oligo d(T) primers, which facilitate hybridization of the cDNA tails to the primers. In a highly-preferred embodiment, cDNA templates are hybridized to oligo d(T) primers and then “locked” into place. Locking is accomplished by the addition of dTTP until all “As” on the polyadenylated tail of the template have a complement. However, because the As and Ts can slide relative to one another, in a second step, a limited number of dATP, dCTP, and dGTP are incorporated into the primer such that the primer and template are prevented from sliding (dissociating). For example, fill and lock can be performed in any of the following ways. In a first embodiment, dTTP and reversible terminator analogs of A, C, and G nucleotide are combined. In this method, the dTTP fill the complement to the poly-A sequence of the template, and the terminators lock the primer and template together such that they cannot slide relative to one another. In a second embodiment, dTTP is added and then washed away, followed by addition of the other 3 nucleotides. Finally, in a third embodiment, all 4 nucleotides are added sequentially starting with dTTP with washing steps following each nucleotide addition. dTTP and 1 nucleotide (e.g. dATP) are added and washed away, followed by dTTP and the next nucleotide (e.g. dCTP) and a wash, and finally the addition of dTTP with the last nucleotide (e.g. dGTP).

In a preferred embodiment, cDNA strands prepared as described above are sequenced using single molecule sequencing. In single molecule sequencing, template (cDNA)/primer duplex are individually optically resolvable on a sequencing substrate. Single molecule sequencing is taught in co-owned U.S. Pat. No. 7,169,560, and U.S. application Ser. No. 10/990,167 (filed Nov. 16, 2004, Publication No. US 2006-0012793 A1), each of which is incorporated by reference herein. Essentially, polyadenylated cDNA is hybridized to poly dT primers attached covalently to an epoxide-coated glass surface. Poly dT primed surfaces and their uses are disclosed in co-owned U.S. patent application Ser. No. 11/958,173 filed Dec. 17, 2007, incorporated by reference herein. After rinsing, the surface-bound duplex is exposed to one or more dNTPs, or analogs, comprising a detectable label, and a polymerase enzyme under conditions sufficient for template-dependent sequencing-by-synthesis. In a preferred embodiment a single species of dNTP is added and in a highly-preferred embodiment, the dNTP is an analog comprising a detectable label and an inhibitor of subsequent nucleotide incorporation, both being attached to the dNTP by a cleavable linker. Upon incorporation, the analog prevents next base incorporation, thus yielding a single incorporation per reaction cycle (assuming the presence of a complementary nucleotide in the template). After a wash step, incorporated nucleotides are visualized and recorded by position on the surface. The linker is then cleaved and duplex are prepared for subsequent cycles of nucleotide addition. Upon completion of a user-determined number of addition cycles, each position on the surface (representing a single duplex) will have associated with it a number of nucleotides representing the sequence of additions (and hence the sequence of the template) at that duplex. Informatic methods, such as those taught in co-owned, U.S. patent application Ser. No. 11/347,350 (filed Feb. 3, 2006; Publication No. US 2006-0286566 A1), incorporated by reference herein, are then used to compile the aligned sequence of the starting material.

Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Substrates are preferably coated to allow optimum optical processing and nucleic acid attachment. Substrates for use in the invention can also be treated to reduce background. Exemplary coatings include epoxides, and derivatized epoxides (e.g., with a binding molecule, such as an oligonucleotide or streptavidin).

Various methods can be used to anchor or immobilize the nucleic acid molecule to the surface of the substrate. The immobilization can be achieved through direct or indirect bonding to the surface. The bonding can be by covalent linkage. (Joos et al., Analytical Biochemistry 247:96-101 (1997); Oroskar et al., Clin. Chem. 42:1547-1555 (1996); and Khandjian, Mol. Bio. Rep. 11:107-115 (1986). A preferred attachment is direct amine bonding of a terminal nucleotide of the template or the 5′ end of the primer to an epoxide integrated on the surface. The bonding also can be through non-covalent linkage. For example, biotin-streptavidin (Taylor et al., J. Phys. D. Appl. Phys. 24:1443 (1991)) and digoxigenin with anti-digoxigenin (Smith et al., Science 253:1122 (1992)) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

In other embodiments, the invention provides methods for nucleic acid transcript analysis comprising synthesizing a cDNA strand from an RNA template using a reverse transcriptase to yield a plurality of cDNA strands of varying read length. The various strands are then sequenced. By controlling the extent of the reverse transcriptase reaction by methods known in the art, the resulting reads will have a variety of start positions. This allows for increase accuracy, especially in long mRNA templates. Controlling the reverse transcriptase reaction also allows more informative counting (i.e., increased variability in start sites leads to more informative counting). The variability in read length introduced by this method also facilitates focus on the most accurate sequencing reads.

According to the invention, transcripts of all lengths are accurately counted when oligo dU or oligo dT are used in the reverse transcriptase reaction. In addition, small-to-average length transcripts benefit from a high representation of full length cDNA facilitating accurate mapping of transcriptional start sites (TSS). The variability in efficiency of the reverse transcription ensures that enough cDNA does not reach full length generating enough reads with start sites spanning the full length of the transcript to provide sequence information. The oligo dT (or oligo dU) RT-priming method thus generates accurate counts, TSS mapping, and full transcript sequence information of around 1500 nucleotides upstream of the 3′ transcript end, all in one application.

The use of a random oligo in the reverse transcriptase reaction provides TSS identification and sequence information for all transcripts regardless of size. The different approaches for long and short transcripts are shown graphically in FIG. 2. Because the enzyme does not reach the 5′ end of longer transcripts, the result is more heterogeneity in the start positions of the various reads, allowing accurate and informative counting across the entire transcript. In the case of shorter transcripts, the enzyme reads through to the 5′ end, allowing a precise determination of the start site.

In another embodiment, the invention provides methods for identifying sequence that is not part of the reference sequence in a sample of transcripts by identifying clusters of unaligned sequences and comparing the unaligned sequences to one or more reference sequences. Thus, according to the invention, unaligned transcript sequencing reads are informative in the identification of new transcripts, contamination, alternative splice variants, and other sequence information not contained in only those sequences that align with a predetermined reference.

In another embodiment, the invention provides methods for counting transcripts, resulting in quantification of expression levels. In one variation of the invention, quantification of expression is used to determine the response of a patient to treatment. In another variation, expression analysis of the invention is used to identify therapeutic targets. In another embodiment, methods of the invention are used to identify transcription start sites, splice variants, and quantification of variants for research and/or clinical analysis. Quantitative methods of the invention are the result of amplification-free sample preparation and single molecule sequencing techniques as described herein.

The digital nature of methods of the invention provide the ability to identify one or more transcriptional start sites in a gene. Methods of the invention also allow for the complete resequencing of transcripts, especially those in moderate to high abundance in a sample, with high coverage and accuracy. Methods of the invention are also useful for the identification of low-abundance transcripts, even if they are relatively short. Methods of the invention also allow for the discovery of splice variants, single nucleotide polymorphisms, mutations, and even new strains/organisms in a sample. Methods of the invention also allow for the identification of viral and/or bacterial infection of a tissue. In principle, the methods of the invention can be used to identify a multiplicity of unknown transcripts in a sample.

Finally, methods of the invention can be combined with statistical and informatic techniques, such as those disclosed in co-owned, U.S. patent application Ser. No. 61/034,138, incorporated by reference herein, in order to further increase the accuracy and reliability of results produced herein.

### BRIEF DESCRIPTION OF THE FIGURES

FIG. 1 is a schematic showing of variation of amplification-free sample preparation. ‘AAAAAAAAAA’ disclosed as SEQ ID NO: 1.

FIG. 2 graphically illustrates the different approaches for long and short transcripts are shown.

FIG. 3 is a schematically illustrate certain embodiments of the invention, particularly with regard to sample preparation, sequencing, and analysis methodology. ‘TTTTTTTACA’ disclosed as SEQ ID NO: 2 and ‘TGTAAAAAAT’ disclosed as SEQ ID NO: 70.

FIG. 4 shows illustrative data, particularly relating to read length and transcript abundance.

FIG. 5 shows illustrative data, particularly relating to reproducibility and counting accuracy.

FIG. 6 shows illustrative data, particularly relating to transcription Start Site mapping.

FIG. 7 shows illustrative data, particularly relating to sequence information.

FIG. 8 shows illustrative sequence characterization. FIG. 8a discloses SEQ ID NOS 3-48, respectively, in order of appearance; FIG. 8b discloses SEQ ID NOS 49-62, respectively, in order of appearance; and FIG. 8c discloses SEQ ID NOS 63-69, respectively, in order of appearance.

FIG. 9 shows illustrative transcription coverage.

FIG. 10 shows illustrative DGE vs RNA-Seq.

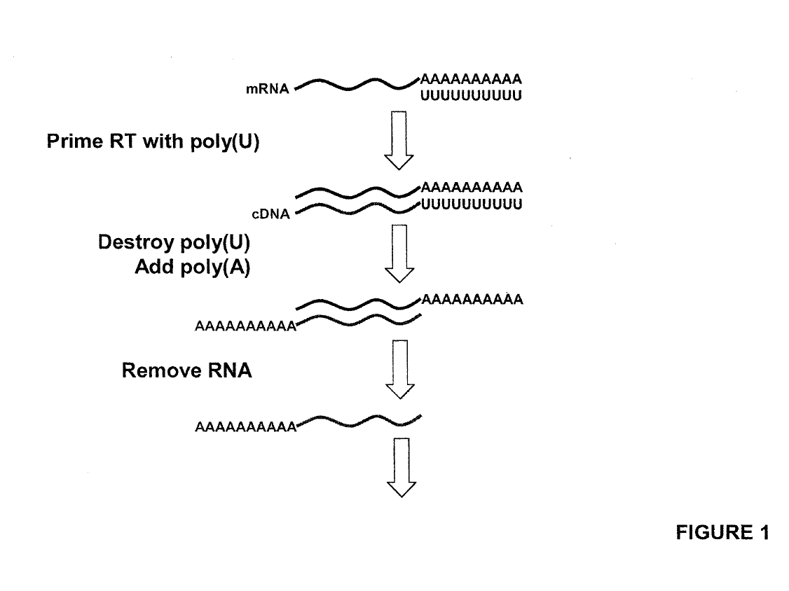

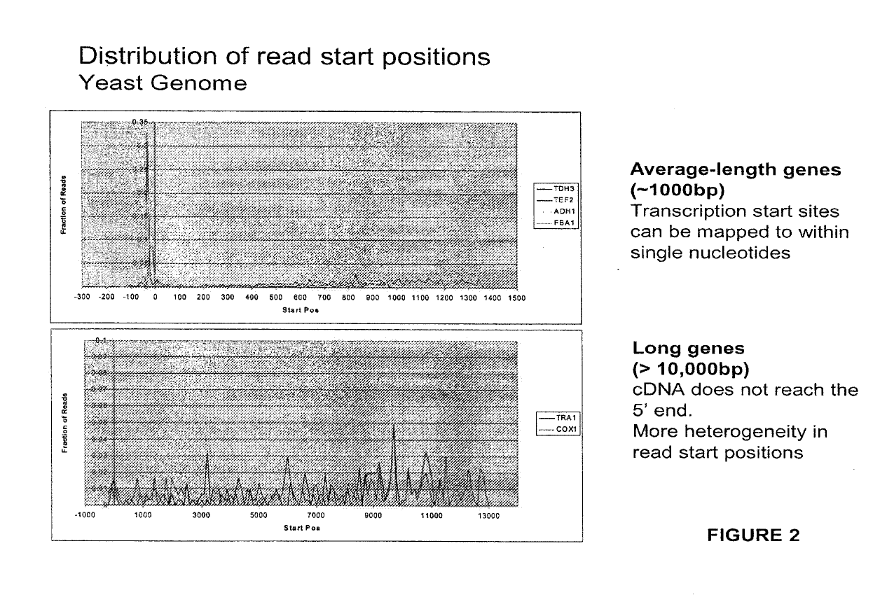

### DETAILED DESCRIPTION OF THE INVENTION

The invention generally provides a unique transcription analysis method, smsDGE, which is a novel transcriptome profiling method utilizing the unique attributes of high-throughput single-molecule sequencing.

Expression profiling by smsDGE overcomes many of the limitations of array-based methods. Specifically, it allows accurate quantification of a wide range of expression levels, including low abundance transcripts. The invention allows detection of sequence variants and it generates counts that are readily comparable between different transcripts, different sample preparations and different runs. In addition, it provides a robust tool for novel discovery such as detection of novel transcripts based on reads that do not align to the known transcriptome reference. smsDGE is based on a simple sample preparation method free of amplification reactions, restriction digest or ligation steps, relying instead on the poly-dA tailing of a cDNA sample by terminal transferase alone. The methods of the invention thereby reduce biases related to preparation steps inherent to previous DGE methods such as SAGE and MPSS9-13.

Short read sequencing technologies have been recently shown to generate quantitative measurement of gene expression via full transcriptome sequencing (RNA-Seq). (Nagalakshmi, et al. Science 320, 1344-1349 (2008); Mortazavi, et al. Nat Methods 5, 621-628 (2008); Cloonan, et al. Nat Methods 5, 613-619 (2008).) RNA-Seq differs from smsDGE by the fact that multiple reads are generated from each transcript molecule, where long transcripts generate more reads in proportion to their length.

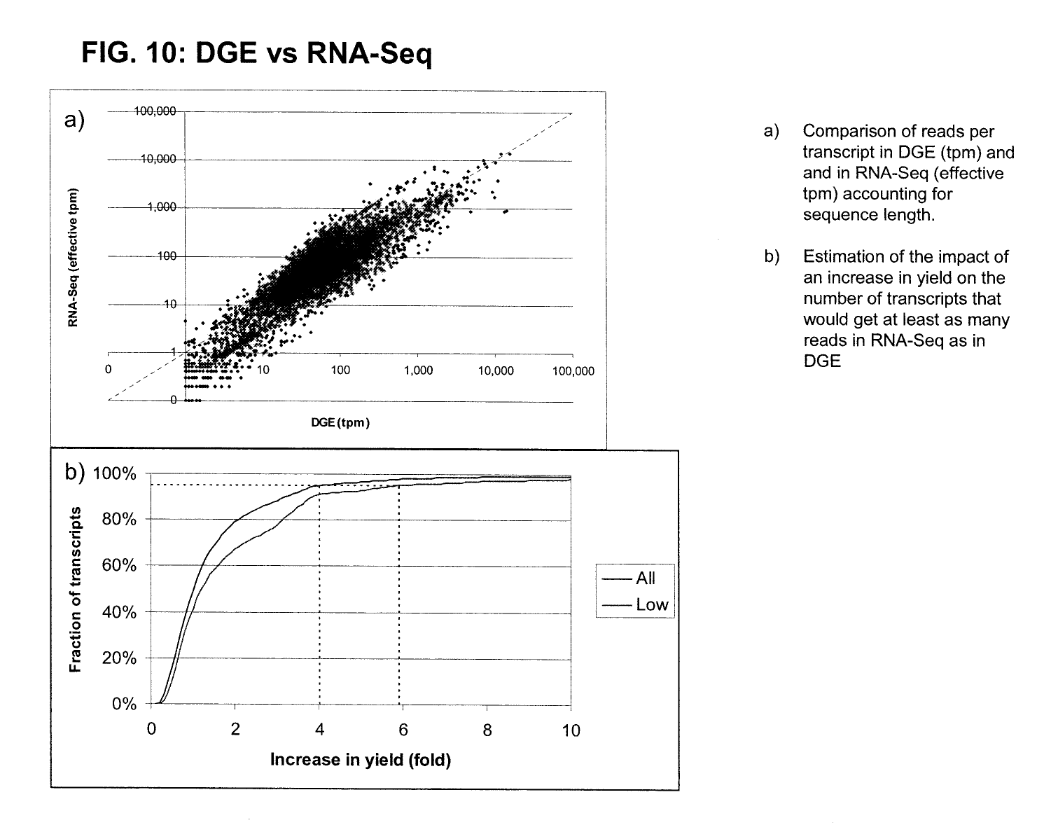

As demonstrated herein, the variance in the measurement of transcript abundance is driven mostly by the expected number of reads that are generated from it. While in smsDGE this variance depends only on transcript abundance, in RNA-Seq, it is dependent on both transcript abundance and length, making short rare transcripts harder to count accurately. The number of observed reads per transcript in smsDGE of the yeast transcriptome are compared with the expected number of reads per transcript that would be generated by RNA-Seq. It would be necessary to generate 40M reads in RNA-Seq to get the same coverage for 95% of transcripts that 10M smsDGE reads would provide (FIG. 10). A similar analysis of transcriptome data from human tissues suggests >5 fold factor. An additional complexity of expression profiling by RNA-Seq is that transcript counts must be derived by a normalization process, that assumes uniform transcript coverage which is hard to achieve (e.g. RPKM15). smsDGE, on the other hand, uses the raw counts directly and is likely to be more accurate in the presence of 3′ biased mRNA material. An additional unique aspect of smsDGE data is that all reads are generated from single stranded cDNA molecules and are therefore strand specific relative to the genome. This is especially advantageous in cases where open reading frames overlap on the forward and reverse DNA strands.

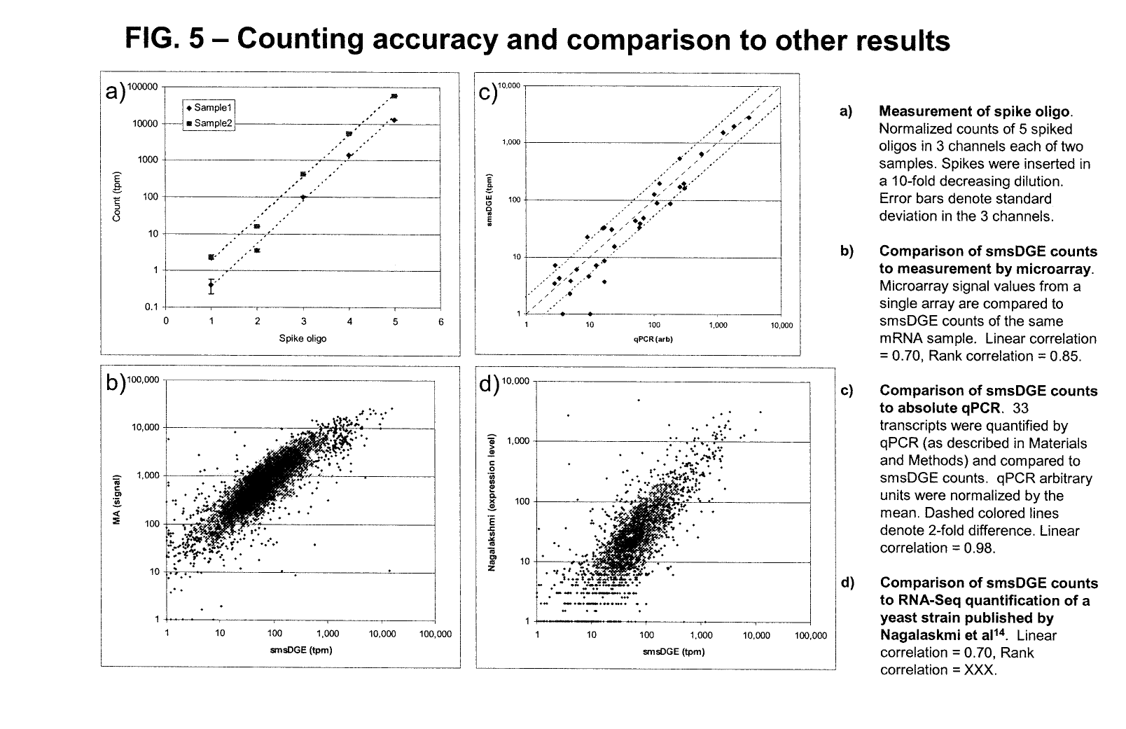

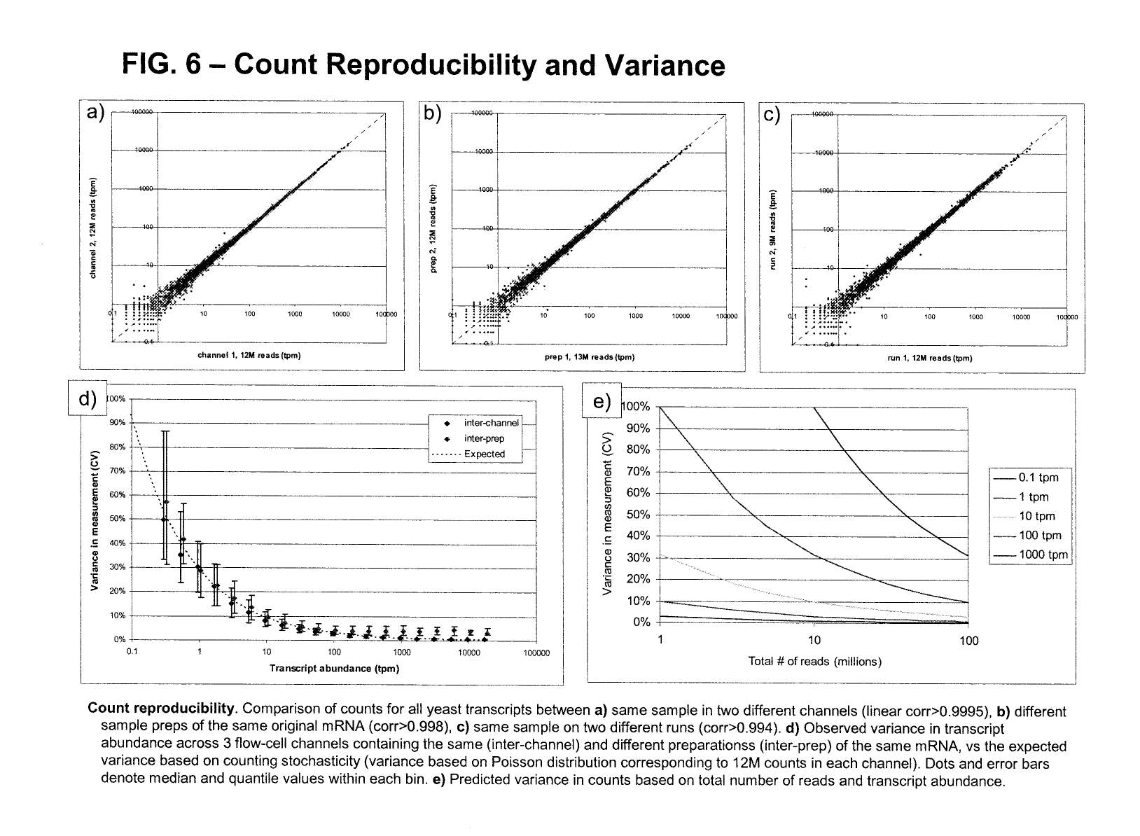

Over 12 million usable (≧24 nt long, transcriptome-aligned) reads, generated in each of 6 channels of the Helicos sequencing platform were used to quantify the complete range of transcripts expressed in the DBY746 strain of S. cerevisiae. Quantification accuracy was assessed using a set of spiked RNA molecules, demonstrating accurate counts across 5 orders of magnitude, and down to an abundance level of below 1 tpm using a single channel (FIG. 5a). High counting reproducibility was demonstrated across different channels, sample preparations and runs (FIG. 6).

Due to the nature of the platform and the variability of the read start sites along each transcript, smsDGE provides a wealth of sequence information covering a significant part of the expressed transcriptome, providing the ability to identify non-annotated transcripts and quantify partially-annotated or divergent transcriptomes. The invention herein provides the ability to discover transcripts that did not appear in the reference library by clustering unaligned reads, and to identify a large number of sequence variants relative to the reference strain. Of independent interest is the capability to map transcription start-sites, especially in low to average sized transcripts.

Here, the simplicity of the yeast transcriptome enabled a clear demonstration of the counting accuracy of smsDGE covering the full cellular dynamic range of this organism. The capacity of smsDGE to provide accurate transcript quantification for a single sample will simplify comparison between independently prepared and measured samples. This ability, combined with the efficiency of transcript counting and the high throughput of the SMS platform will provide cost-efficient expression profiling for large multi-sample studies.

In one aspect, the invention generally relates to a method for analyzing RNA transcripts. The method includes sequencing a first strand cDNA via single-molecule sequencing thereby obtaining transcript information, wherein the method does not comprise a step of RNA or cDNA amplification. In certain embodiments, the method does not comprise RNA or cDNA fragmentation. In certain detailed embodiments, the method includes the steps of: copying a RNA to form a cDNA; polyadenylating the cDNA; hybridizing the polyadenylated cDNA to a primer bound to a surface; and conducting single-molecule sequencing of the cDNA.

The invention provides methods for RNA sample preparation and transcriptome analysis. Methods of the invention provide simple and accurate sample preparation that does not require fragmentation or amplification of sample. Methods of the invention also provide for digital analysis and counting of transcripts that leads to identification of transcription start sites, splice variants, unknown transcripts, mutations, SNPs, and the like. Methods of the invention also make use of unaligned transcript sequences in order to identify contaminants and/or new transcripts.

In preferred embodiments, samples of mRNA are prepared by priming with oligo dT and subsequent synthesis of a complementary DNA strand (cDNA). The cDNA is isolated, polyadenylated, and hybridized to a poly dT primer. Then, the cDNA is sequenced by template-dependent nucleotide addition to the 3′ end of the primer. In preferred embodiments, cDNA/primer duplex are individually optically resolvable and sequencing is carried out using optically-detectable nucleotide analogs on a cyclic basis, such that, on average, only one nucleotide is added to a primer per addition cycle until sequencing is complete.

Read clustering can be used for digital expression analysis even when the reference sequence of the measured sample is unknown. In this context clustering could be used to detect and quantify any sufficiently expressed transcript in the sample, for either absolute transcript quantification in a single sample or differential analysis between multiple samples.

### EXAMPLES

### Example 1

First strand cDNA was made from S. cerevisiae mRNA via oligo-dT priming (Invitrogen SuperScript III kit according to manufacturers instructions). The resulting cDNA was polyadenylated at its 3′ end to yield approximately 50 dATPs. An aliquot of 20 ng of the cDNA sample was combined with KOAc (50 mM), tris base (20 mM), MgAc (10 mM) (for a final concentration of 10%), CoCl (250 μM), dATP (50× the sample molarity), an R110-labeled degradable control oligo used to assess the tailing efficiency (0.5 pmole). The reaction was denatured at 95° C. for 5 minutes and quickly chilled on ice for an additional 2 minutes. 20 U of terminal transferase were then added to the sample mix and incubated at 42° C. for 1 hour followed by a 10 minute enzyme heat inactivation step (70° C.). The polyadenylated cDNA was then hybridized to a surface comprising oligo dT primers (50-mers) as described in co-owned, U.S. Pat. No. 7,282,337, incorporated by reference herein. Sequencing-by-synthesis was carried out for thirty 4 nucleotide addition cycles. The resulting sequence reads collected were then identified by alignment to the S. cerevisiae transcriptome reference. Each read was representative of a single molecule. The variation in cDNA length resulting from RNA degradation, or reverse transcriptase incomplete transcription, allowed for complete sequencing coverage of more highly expressed mRNAs. Approximately 1 million alignable reads were collected; allowing for expression detection approaching 10 tpm. Reads were aligned via a statistical counting method (e.g., as described in PCT/US09/30952 filed Jan. 14, 2009, which is incorporated by reference herein) using an error-tolerant read seeding method.

Complete alignment of transcript reads were obtained by methods of the invention with the S. cerevisiae TDH3 open reading frame at 60× coverage.

### Example 2

In a separate experiment, sequencing reads were obtained as described above from human placenta mRNA sample. Those that did not align to the relevant human placenta reference sequence were clustered based upon sequence similarity and the consensus sequence of the cluster was aligned to the complete NCBI sequence database using BLAST. The sequence was identified as a highly-significant match to an MHC class I antigen from S. scrofa. This is likely a contaminant introduced in sample preparation.

Further methods and embodiments of the invention are apparent to the skilled artisan upon review of the present disclosure.

### Example 3

### Methods

cDNA preparation

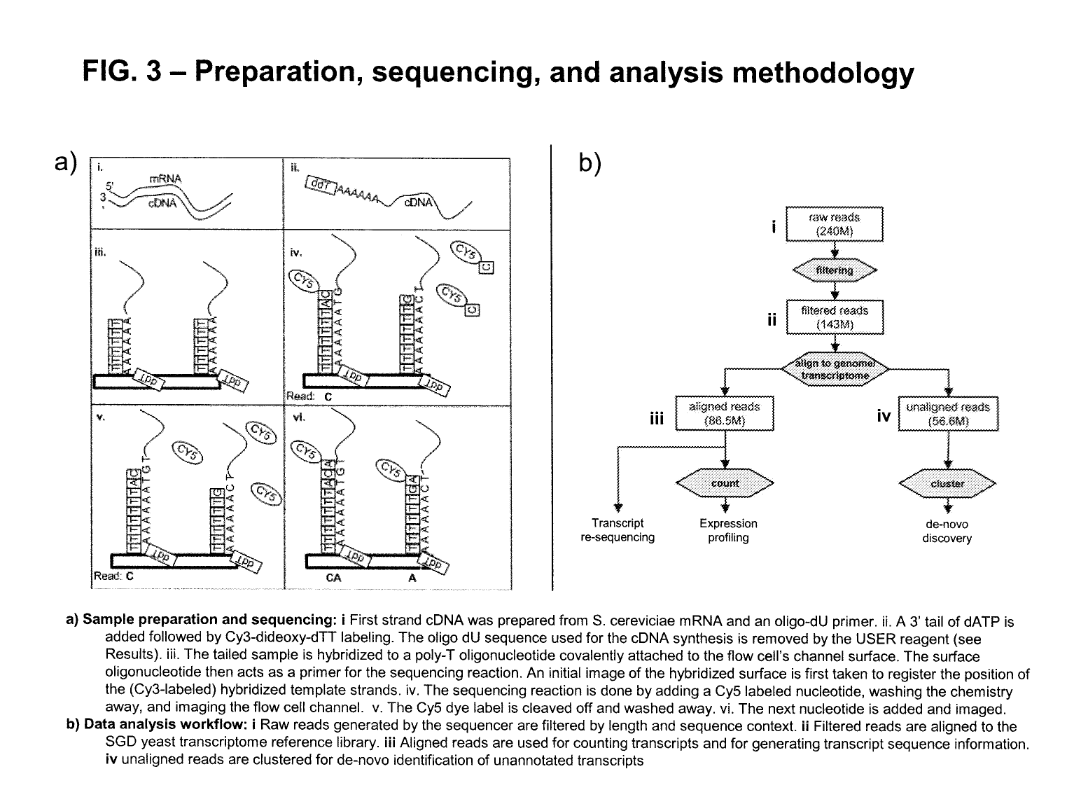

mRNA from S. cerevisiae strain DBY746 (his3Δ1 leu2-3 leu2-112 ura3-52 trp1-289), grown under standard conditions (YPD, 30oC) was obtained from Clontech (Mountain View Calif.). 1 μg S. cerevisiae RNA was mixed with 6 in-vitro transcribed Arabidopsis thaliana RNAs at 40 ng to 400 fg as described in FIG. 3 legend (Stratagene, Agilent technologies La Jolla Calif.). In addition 3 assay replicates were prepared independently from the same RNA for assay reproducibility studies. 1 to 2 μg yeast poly A selected RNA was used to make first strand cDNA. First strand cDNA was prepared using a SuperScript III first strand cDNA synthesis kit (Invitrogen, Carlsbad Calif.) according to manufacturers instructions except that 5 μM of a 50 nucleotide deoxyuracil primer (IDT, Iowa City Iowa) was used in place of the recommended primer. mRNA was removed by RNase H (Invitrogen, Carlsbad Calif.) digestion for 20 min. at 37° C. followed by removal of the deoxyuracil primer sequence by USER Reagent (New England Biolabs, Ipswich Mass.) digestion for 20 min. at 37° C. A final incubation with RNase I (New England Biolabs, Ipswich Mass.) for 15 min. at 37° C. was then performed to remove any remaining RNA. The sample was purified using the AMPure kit (Agencourt Biosciences, Beverly Mass.) at a 1:1.8 sample to bead ratio according to manufacturer's instructions. The above preparations yielded approximately 500 to 1000 ng cDNA for 1 and 2 μg preparations respectively. 60 ng of this prepared cDNA was then poly dA tailed and loaded on 1-3 channels of the HeliScope sequencer.

### Poly dA Tailing

A poly dA tail of 90±20 nucleotides on average was added to the 3′ end of the cDNA by terminal deoxynucleotidyl transferase (New England Biolabs, Ipswich Mass.). A 60 ng cDNA sample was combined with terminal deoxynucleotidyl transferase reaction buffer (KAc (50 mM), tris acetate (20 mM), Mg Acetate (10 mM), pH 7.9) CoCl₂ (250 μM), dATP (170 pmoles), and a control oligo used to assess the tailing efficiency (1.5 pmole). 24 units terminal transferase were added after denaturation and snap cooling on ice, followed by 1 hour incubation at 42° C., and 10 min. heat inactivation at 70° C. Tailed samples were then labeled and 3′ blocked by dideoxy TTP (600 pmoles). The sample was then denatured and snap cooled on ice, 24 units of terminal transferase were added followed by a 1 hour incubation at 37° C. and a final heat inactivation step. The control oligo and excess nucleotides were removed from the sample by Ampure purification at a 1:1.3 sample to bead ratio (Agencourt Bioscience, MA).

### Template Capture and Sequencing

Each sequencing reaction takes place in one of 50 channels of the sequencing flow cell. Each channel's surface is lined with a covalently attached Poly-dT oligonucleotide. This surface oligonucleotide has the dual role of facilitating the template capture and priming the sequencing reaction. For capture, the cDNA template's poly-dA 3′ tail is hybridized to the poly-dT surface oligonucleotide. The sequencing reaction can then be initiated at the surface oligo's 3′ end (see FIG. 3). To avoid sequencing of the template poly-dA tail, sequencing is preceded by a ‘fill and lock’ procedure in which the surface oligo is extended against the template's 3′ poly-dA tail by a dTTP fill. dGTP, dCTP, and dATP VTs are also included in the reaction to ‘lock’ the surface oligo against the sample template after the dTTP fill is complete (1 μM dTTP, DNA polymerase I and 1×NEB buffer 2; NEB, MA).

Sequencing by synthesis is performed following the ‘fill and lock’ procedure by introducing one of four Cy5 labeled VT nucleotides in the presence of a polymerase reaction mix. Incorporated nucleotides are imaged after which the Cy5 dye is chemically cleaved off the incorporated nucleotide and rinsed away. This process is repeated for each of the next 3 nucleotides to complete a sequencing quad cycle. A total of 30 quad cycles were preformed. The process of sequence base calling was previously described and used here with the exception that no intensity based homopolymer length calling was performed in this study since VT nucleotides do not run through homopolymer sequences.

### Quantitative PCR

33 S. cerevisiae transcripts spanning a large range of expression levels were selected for comparison of smsDGE counts against qPCR quantification (18 Taqman and 15 SYBR green assays). 13 of these 33 transcripts were selected from transcripts with smsDGE counts <10 tpm to test accuracy at low abundance levels. qPCR reactions were denatured at 95° C. for 10 min. followed by 40 cycles of 95° C. for 15 s and 57° C. for 30 s. Taqman assays had forward and reverse primers at 0.3 μM each, a Taqman probe at 0.25 μM and 1× Taqman reaction mix (Taqman universal PCR mix, Applied Biosystems, CA). SYBR green assays had forward and reverse primer at 0.15 μM each, and 1×SYBR green mix (Invitrogen, CA).

qPCR normalization was done in two steps: (1). Each transcript was first quantified using a yeast genomic DNA standard. (2). Quantification was then standardized against an arbitrarily selected reference transcript—YDL047W. In 13 out of 33 of the more abundant transcripts quantification was done against YDL047W alone.

### Data Analysis

Data analysis and computational methods, including processing of reads, alignment, transcript counting, detection of sequence variants and clustering, are described in detail in the Supplement.

### Sample Preparation and Sequencing

Sequencing was performed on a Helicos™ Genetic Analysis system. The system's basic design was described in detail by Harris et al. (Harris, et al. Science 320, 106-109 (2008).) Additional improvements, such as novel nucleotide chemistry and the smsDGE assay methodology are detailed below. The Helicos™ sequencer allows separate sequencing reactions to take place in two flow-cells each consisting of 25 sequencing channels, thereby enabling 50 samples to be sequenced in parallel. The sample preparation procedure and sequencing reaction is overviewed in FIG. 3a. Briefly, mRNA from S. cerevisiae strain DBY746, grown under standard conditions, was used for first strand cDNA synthesis and a poly-dA tail was added to the 3′ end of the single-stranded cDNA. The sample was then hybridized to poly-dT oligonucleotides covalently attached to the surface of a flow-cell channel. This hybridization allows the attached oligonucleotides to be used as primers for the subsequent sequencing reaction. Sequencing is achieved by sequentially incorporating fluorescently-labeled Virtual Terminator™ (VT) nucleotides. These nucleotides allow incorporation of only a single nucleotide at a time onto the growing sequenced strand, preventing homopolymer run-through. (e.g., PCT International Application No. PCT/US08/59446 filed Apr. 4, 2008, U.S. application Ser. No. 12/098,196 filed on Apr. 4, 2008 and U.S. application Ser. No. 12/244,698 filed Oct. 2, 2008) Sequencing information is then attained by laser illumination imaging of the surface and recording of nucleotide incorporations at each DNA strand location. The serial incorporation and imaging of all four nucleotides is termed a “quad-cycle”; 30 quad-cycles were used for the S. cerevisiae transcriptome profiling described here. Two independently prepared samples from a single source of mRNA were run in 3 separate flow-cell channels each.

### Data & Alignment

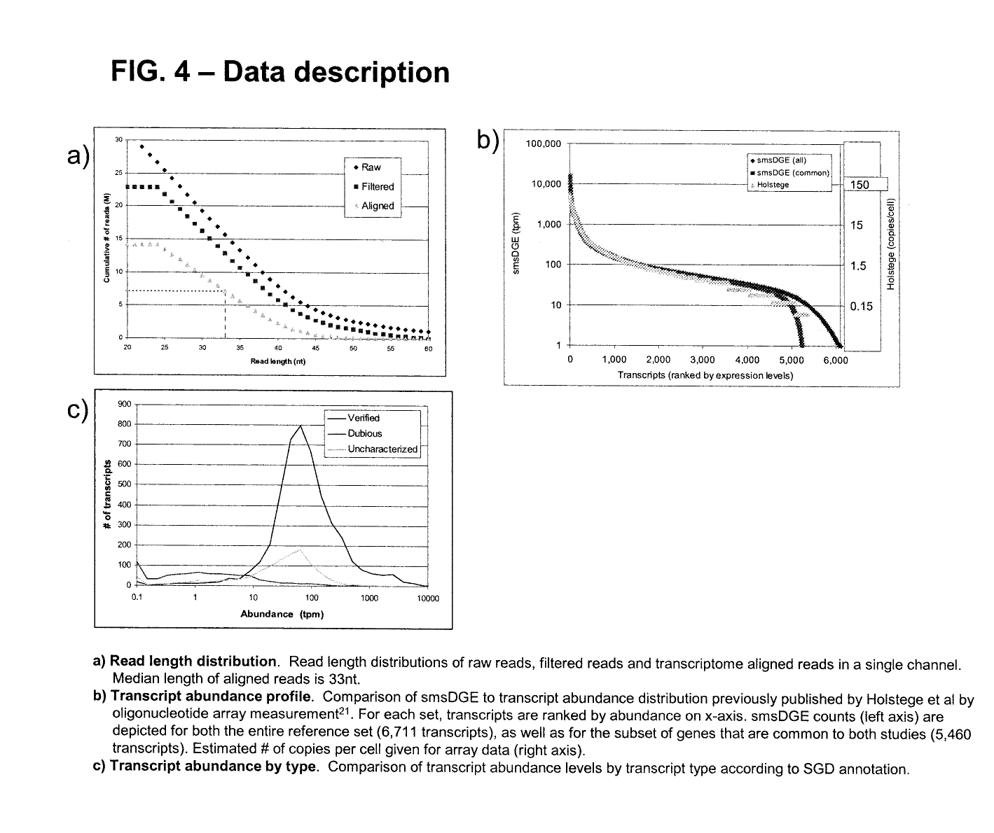

The data analysis workflow is outlined in FIG. 3b. An initial 240M raw reads were collected from 6 channels of a single run. Filtering by length and sequence complexity yielded a final count of 143M reads of 24-60 nt in length (60% of raw reads, where the attrition is mostly attributable to the minimal length criteria). Reads were aligned to both a complete S. cerevisiae genome reference and a transcriptome reference library consisting of single-stranded 5′ UTR and ORF sequences of 6,719 verified, uncharacterized and dubious ORFs from the Saccharomyces Genome Database (SGD). (Fisk, et al. Yeast 23, 857-865 (2006).) Short read alignment was performed using a Smith-Waterman based alignment algorithm, which is tolerant of indel errors, using a stringent threshold. In total, 86M (60%) of the filtered reads could be mapped to the yeast genome at the given stringency, and 78M (55%) could be mapped to at least one yeast transcript. The high fraction of reads mapping to the transcriptome (91%) is indicative of the relative completeness of the yeast transcript annotation, where the remaining 9% of mapped reads are attributable to reads derived from spurious reverse strands and unannotated transcripts. The aligned read length distribution spanned the entire range of 24-60 nt, where >99% of reads were length 24-50 nt with a median length of 33 nt (FIG. 4a). Since read growth rate varies by sequence context (resulting from the order in which bases are added in the sequencing reaction) 30 quad-cycles were used to ensure that slow growing reads could reach the threshold length. The average error rates, based on reads mapped with high confidence, were in the range of 4.4-4.8% errors per read base across the 6 channels. The set of reads generated in this study is provided as supplement data of this publication.

### Transcript Counting

A transcript distribution based on short tags is typically derived from a unique assignment of each read to a single transcript. However, due to the occurrence of natural transcript sequence homologies, sequence variance and read errors, unique assignments based on best alignment scores may lead to miscounting due to ambiguous or incorrect assignment. A method for assigning reads that match equally well to several sites ('multireads') has been reported, (Mortazavi, et al. Nat Methods 5, 621-628 (2008)) but does not account for suboptimal-scoring alignments which is significant when considering misassignment between transcripts of radically different abundances. Read misassignment to abundant transcripts will not significantly skew transcript counts. However, low abundance (or non-existant) transcripts will be over-counted since the number of misassigned reads may be on the order of the correct assignments. To achieve maximal assay specificity, a probability-based method was employed for assignment of reads to transcripts. Read Misassignment Corrected counting (RMC-Counting), is described in detail in the Supplement Methods. Briefly, suboptimal alignments between each read and the entire reference library are considered, and the probability of assignment of each read to each transcript is assessed based on both the alignment score and the transcript abundance. Since the latter value is initially unknown, it is derived iteratively based on some initial assessment. Finally, reads that have a significant probability of having been misassigned (the best alignment having a high probability of being incorrect) are discarded, and the vote of ambiguously aligned reads is distributed among all respective transcripts based on their assessed abundances. A final tally of counts assigned to each transcript is reported as transcripts per million (tpm). Since only one read is generated per transcript molecule, transcript length normalization is not applied.

The smsDGE profile of the S. cerevisiae transcriptome is depicted in FIG. 4b. 6,086 (91%) transcripts of the 6,711 putative ORFs in the reference set were measured at an abundance of 1-16,000 tpm, and 5,376 (80%) at a level of >10 tpm.

This profile demonstrates high agreement with a transcript level profile previously measured for 5,460 genes using oligonucleotide arrays. (Holstege, et al. Cell 95, 717-728 (1998).) In addition, this comparison demonstrates that smsDGE transcript counts span at least 4 orders of magnitude of expression levels (defined as 0.01-100 transcripts per cell by Holstege et al.) with higher resolution of low abundance transcripts than was demonstrated in the microarray study. The remaining 625 (9%) of the ORFs in the reference set were detected at a level of <1 tpm, signifying extremely low or no expression. Amongst these are 393 ORFs annotated in SGD as “dubious”, and only 62 ORFs annotated as “verified”. This infrequent detection of transcripts described as dubious serves as a validation of the high specificity attainable by this method (see FIG. 4c).

To demonstrate accurate quantification of low abundance transcripts and assess the dynamic range of transcript detection, five synthetically generated RNAs were serially diluted across 4 orders of magnitude, and mixed with two samples of S. cerevisiae poly-A selected RNAEach RNA sample was then prepared separately and sequenced in three channels. Quantification of the mixed spike RNAs was highly linear ranging from 0.5 to 50,000 tpm demonstrating accurate quantification within each channel with a dynamic range of 4 orders of magnitude, and high reproducibility among the channels (FIG. 5a).

smsDGE counts were compared to a microarray analysis of the identical S cerevisiae sample (Affymetrix Yeast 2.0 Array, performed by Expression Analysis, NC) and assessed the correlation between smsDGE counts in a single channel and unprocessed microarray signal levels (FIG. 5b). Absolute transcription profiling by microarrays is known to be inaccurate due to probe heterogeneity, and the measured absolute signal levels are expected to vary within an order of magnitude. The agreement between the array intensity signal and smsDGE measurements indeed follows this pattern, demonstrating an overall correlation of 0.70 (rank correlation of 0.85; linear correlation is negatively affected by the non-linear saturation of the array signal). In addition, smsDGE counts were compared to qPCR measurements of the same mRNA sample on a panel of 33 transcripts at a wide range of transcription levels (FIG. 5c).

This comparison demonstrates a particularly high correlation (r>0.98, p<10-20) of smsDGE counts, covering over 3 orders of magnitude. 30 out of 33 transcripts (91%) fell within a 2.5-fold range of their respective qPCR measurements. The 3 outliers were transcripts that were measured by smsDGE at lower levels than the respective qPCR measurements, at relatively low abundance levels (<4 tpm). Interestingly, one of these outliers was found to overlap with a large number of reads found on the opposing DNA strand, suggesting that the higher abundance of this amplicon measured by qPCR is in fact, a result of the inability of qPCR to distinguish between transcripts on both strands. The other outliers could be due to under-counting by smsDGE, or over-detection by qPCR (e.g due to cross-hybridization).

### Counting Reproducibility

Counting results were highly reproducible between different flow-cell channels for each sample (corr>0.9995 for all channel pairs, FIG. 6a). Reproducibility was only marginally lower between the two different sample preparations in the same run (corr>0.998, FIG. 6b), and the same sample on two separate runs (corr>0.994, FIG. 6c) suggesting the smsDGE counts are comparable between different preparations and sequencing runs.

To assess counting variability across independently prepared samples a third S. cerevisiae sample was prepared from the same RNA used for the 2 samples described above (FIG. 6d). Inter-sample variability is only slightly greater than the expected sampling stochasticity (of a Poisson sampling process), and is mostly observable at high expression levels (since high abundance transcripts have negligible sampling-based variance). With 12 M reads per channel, the median CV at 100 tpm is 4%, at 10 tpm it is 10% and at 1 tpm—30%. The predictability of the count variance allows us to forecast the effect of additional throughput on counting accuracy, and to determine the minimal number of reads required to reliably detect changes in transcripts of given abundance (FIG. 6e).

### Transcription Start Site Mapping

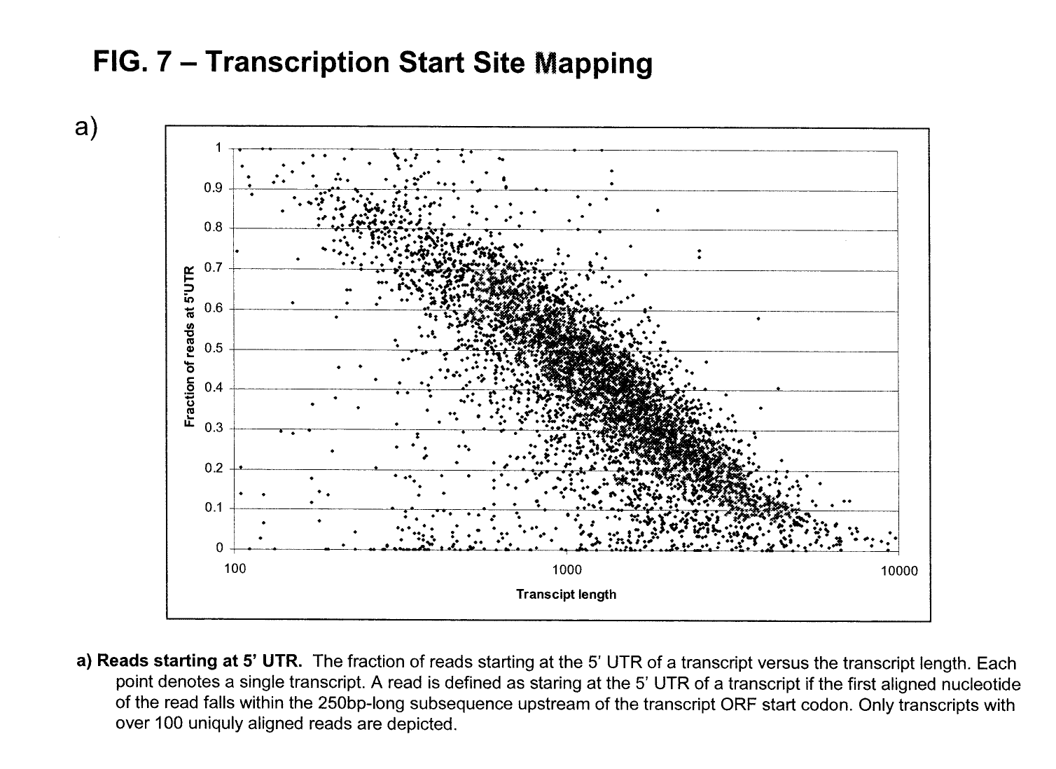

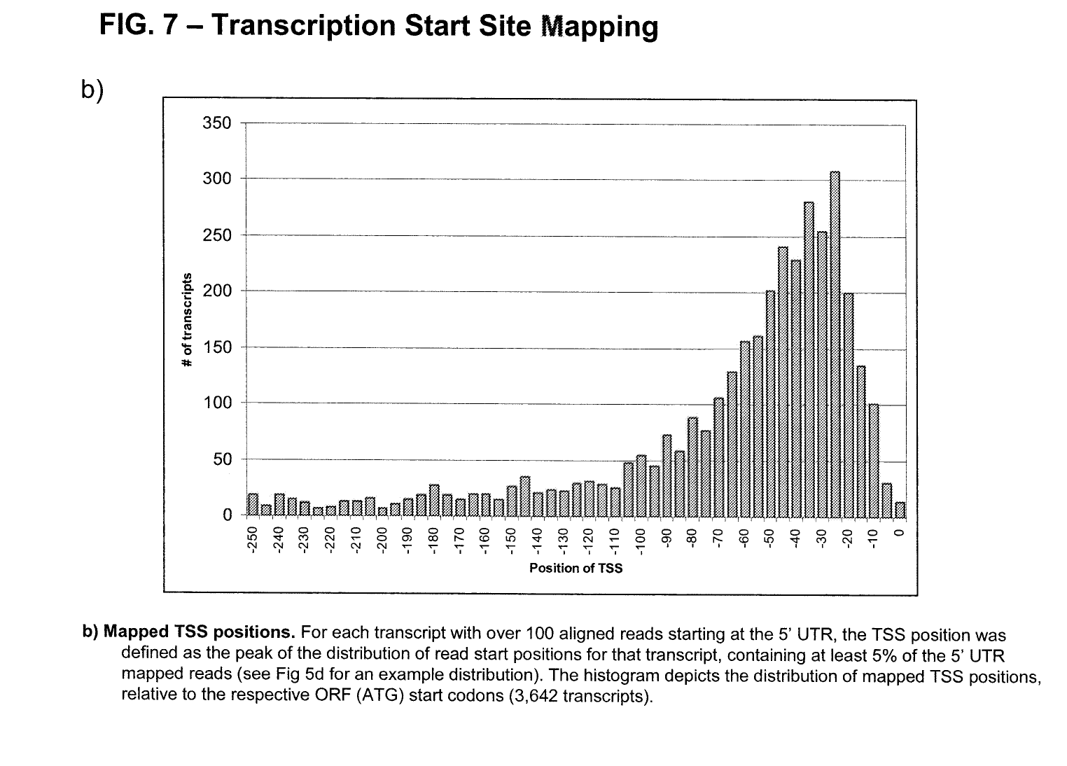

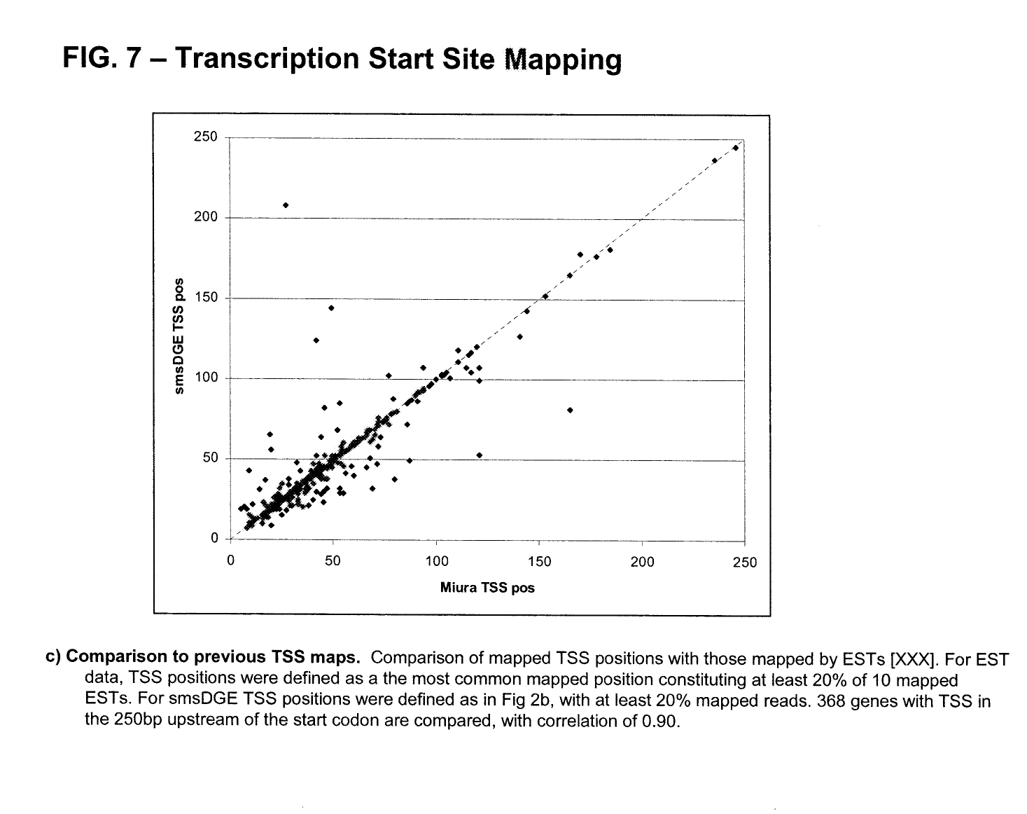

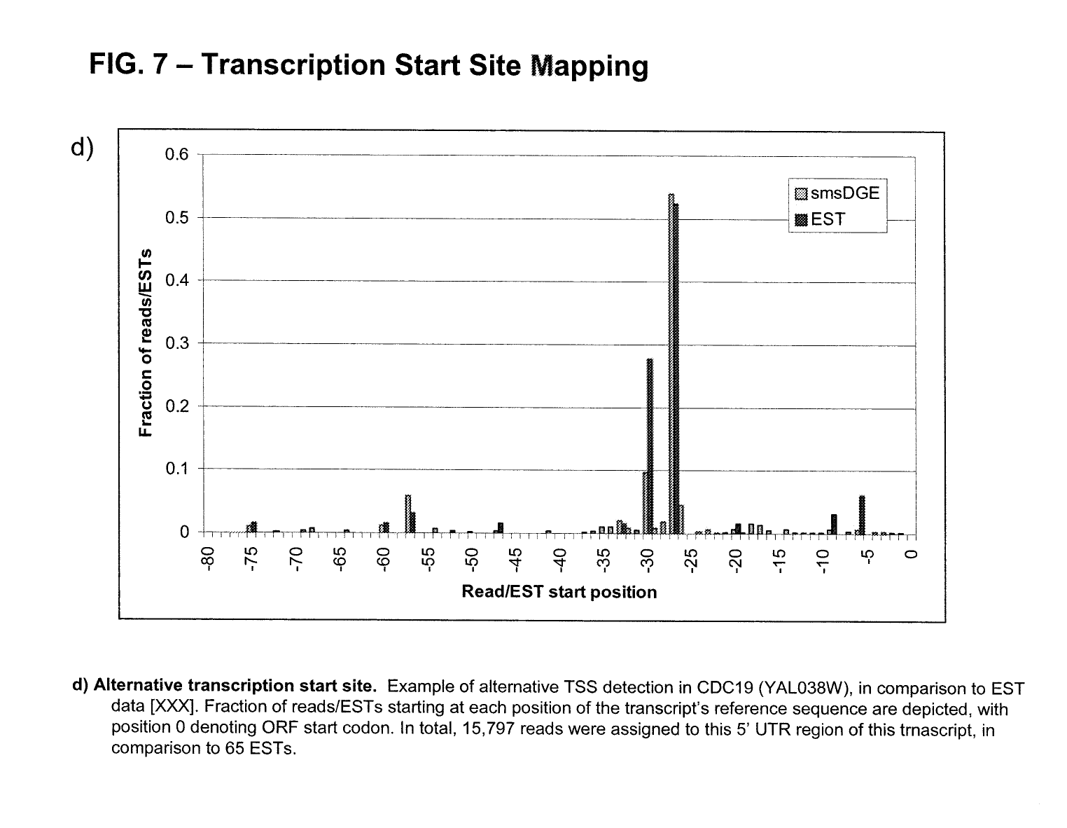

This study provides an excellent opportunity to view the transcriptional start site (TSS) of genes due to the significant number of reads sequenced from the 5′ end of complete transcripts. To allow mapping of reads to 5′ UTR regions, our reference transcriptome library included the additional sequence up to 250 bp upstream of the ORF start codon. In total, 55% of the reads uniquely mapped to 5′ UTR regions, 72% of which begin at the region 50 bp upstream of the ORF start codon—the assumed TSS position of most yeast transcripts 20, 21. As expected, due to limited reverse transcriptase processivity and/or mRNA degradation, the fraction of reads reaching the 5′ end of a transcript is inversely proportional to length (FIG. 7a). Previous efforts to accurately map TSS of the yeast transcriptome by 5′ SAGE, and EST sequencing provided significant information. (Zhang, et al. Nucleic Acids Res 33, 2838-2851 (2005); Miura, et al. Proc Natl Acad Sci USA 103, 17846-17851 (2006).) The data generated in this study enhances these results by providing tens to thousands of reads per TSS for many transcripts, allowing accurate mapping of the physical TSS (over 4,100 transcripts have >100 reads uniquely mapping to their 5′ UTR, in a single channel). FIG. 7b demonstrates the distribution of mapped TSS positions, relative to the respective ORF start codons. FIG. 7c depicts an example of a transcript with multiple alternative TSS positions which are in agreement with mapping data previously described for this transcript. (Miura, et al. Proc Natl Acad Sci USA 103, 17846-17851 (2006).)

### Additional Transcript Characterization

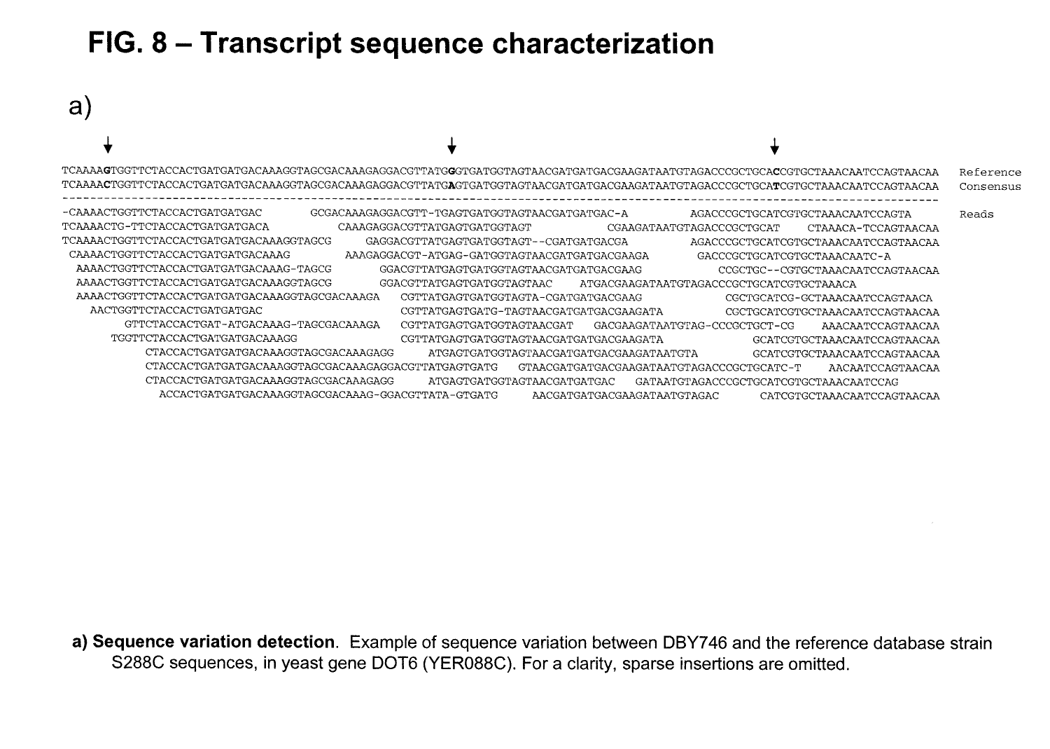

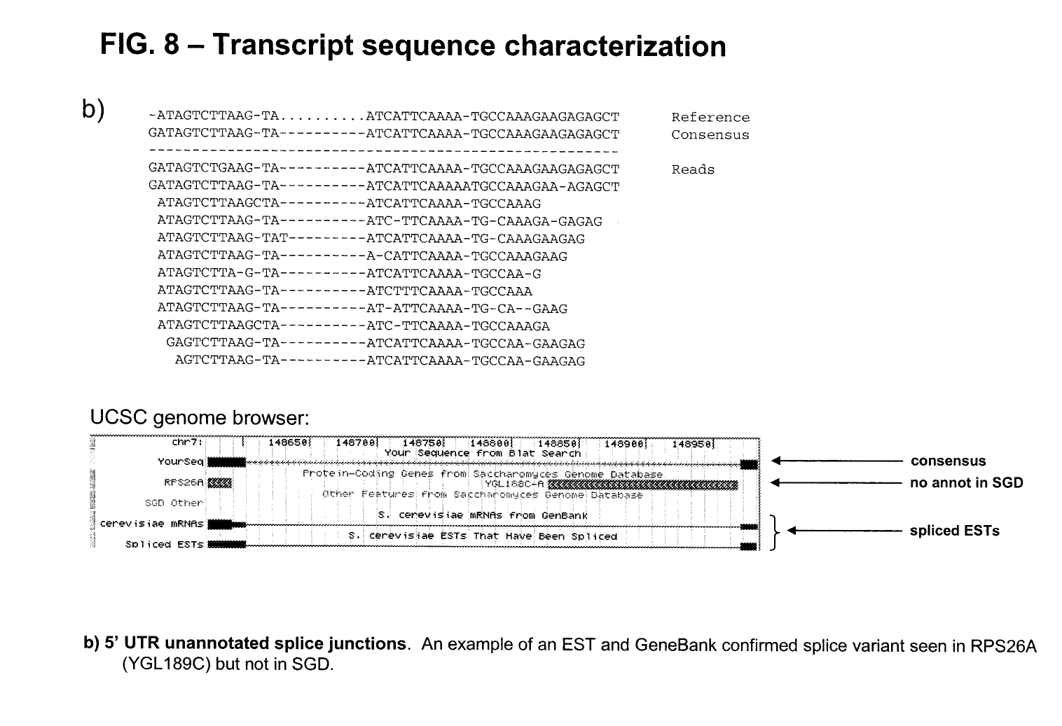

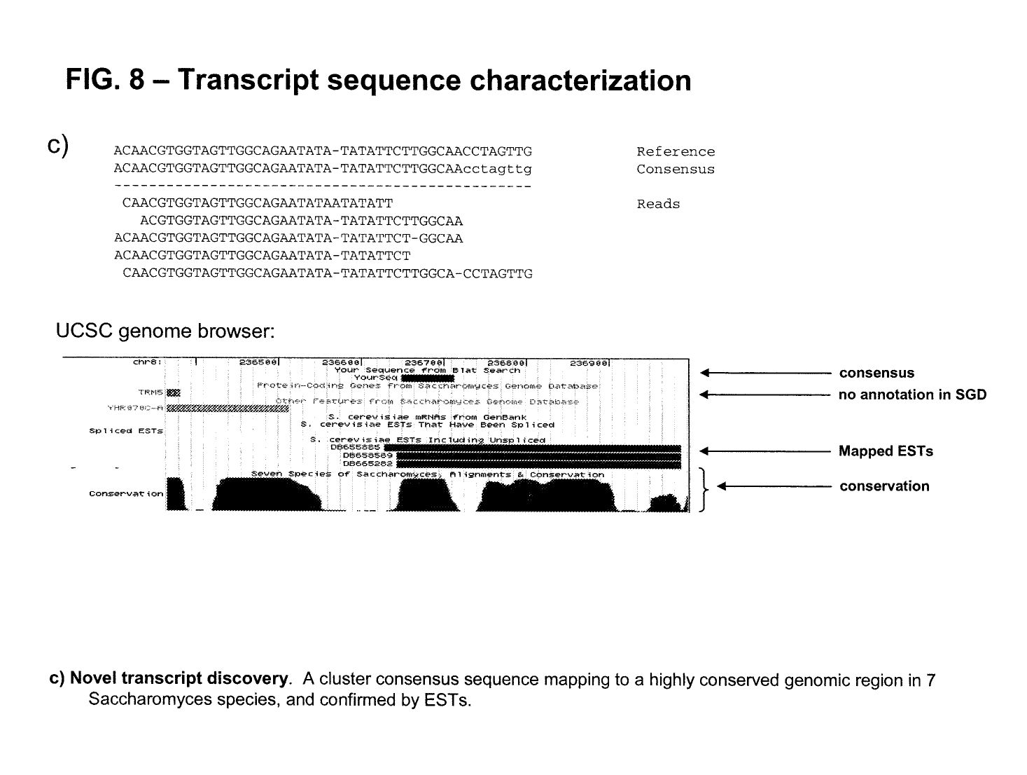

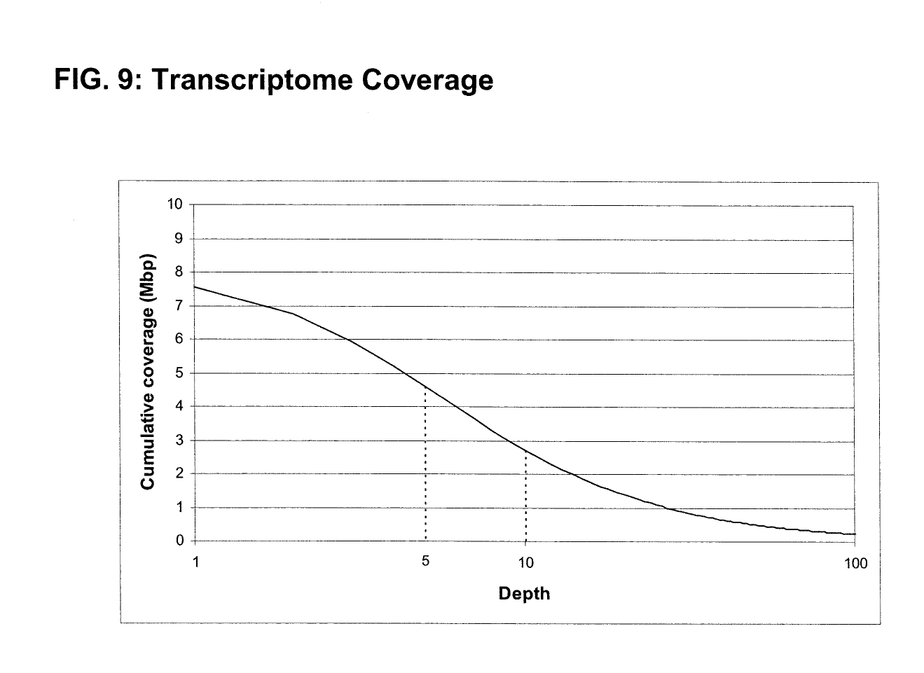

Although the primary goal of this study was to demonstrate the ability of smsDGE to provide accurate abundance levels of all yeast transcripts, the variability in read start sites for each transcript provides a wealth of transcriptome sequence information. This variability is the result of the presence of cDNAs that did not reach full length due to incomplete reverse transcription. The sequence coverage of transcripts varies significantly as a result of their relative sizes and abundance levels, and is typically non-uniform across any single transcript. However, as depicted in FIG. 9, uniquely aligned reads from a single channel covered 7.6 Mbp (84%) of the ˜9 Mbp of S. cerevisiae transcriptome coding sequence, with 4.6 Mbp (51%) covered at a depth of ≧5x, and 2.7 Mpb (30%) at >10x. Applying a simple SNP discovery tool to these reads Over 3,000 single-base substitutions were identified between the strain being sequenced (DBY746) and the strain in the reference database (S288C). FIG. 8a demonstrates three sequence variations in DOT6.

To identify unknown transcripts in the sample the filtered reads were aligned against the complete S. cerevisiae genome. 700K reads mapped to intergenic regions that are further than 250 bp from annotated ORFs. 370K of these reads could be grouped into 1,049 peaks with expression levels over 5 tpm, many of which could be associated with annotations such as distant spliced 5′ UTRs, rRNAs and snRNAs, while others did not match any annotation. As an example, FIG. 8b depicts one of the peaks mapping to an unannotated genomic sequence in agreement with published ESTs and in a region highly conserved among seven yeast species. (Miura, et al. Proc Natl Acad Sci USA 103, 17846-17851 (2006); Kent, et al. Genome Res 12, 996-1006 (2002).) An mRNA sample may include additional non genome-alignable transcripts, such as contaminants or spliced or edited RNA. To demonstrate the ability of smsDGE for de-novo characterization of unknown sequences, a read-clustering strategy was employed to a subset of reads poorly aligned to either the genome or transcriptome libraries. 40,000 reads of length>30 nt were arbitrarily selected and all pairwise alignments between reads were calculated. A variant of the CAST clustering algorithm was used to identify clusters of reads that have a high degree of mutual similarity. (Ben-Dor, et al. J Comput Biol 6, 281-297 (1999).) The consensus sequence for each of these clusters was then calculated and mapped to the non-redundant NCBI database using BLAT. Twenty-two consensus sequences could be mapped to 5′ UTR splice junctions that were previously discovered by EST mapping and tiling arrays (e.g. FIG. 8c). (Miura, et al. Proc Natl Acad Sci USA 103, 17846-17851 (2006); Juneau, et al. Proc Natl Acad Sci USA 104, 1522-1527 (2007).)

### Incorporation by Reference

References and citations to other documents, such as patents, patent applications, patent publications, journals, books, papers, web contents, have been made in this disclosure. All such documents are hereby incorporated herein by reference in their entirety for all purposes.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples herein and the references to the scientific and patent literature cited herein. The examples contain important additional information, exemplification and guidance which can be adapted to the practice of this invention in its various embodiments and equivalents thereof.

What is claimed is:

1. A method for analyzing RNA transcripts, the method comprising sequencing a first strand cDNA via single-molecule sequencing thereby obtaining transcript information, wherein the method does not comprise a step of RNA or cDNA amplification.

2. The method of claim 1, wherein the method does not comprise RNA or cDNA fragmentation.

3. The method of claim 1, wherein the method comprising the steps of:
copying a RNA to form a cDNA;
polyadenylating the cDNA;
hybridizing the polyadenylated cDNA to a primer bound to a surface; and
conducting single-molecule sequencing of the cDNA.

4. The method of claim 3, further comprising obtaining a RNA sample from a tissue or body fluid of a subject.

5. The method of claim 4, wherein the subject is a human.

6. The method of claim 1, wherein the RNA transcripts are from a human gene.

7. The method of claim 3, wherein the surface comprises a glass surface.

8. The method of any of claims 1 to 7, wherein the RNA or cDNA is from about 20 nt to about 500K nt.

9. The method of claim 8, wherein the RNA or cDNA is from about 100 nt to about 100K nt.

10. A method for detecting a sequence in a sample that does not align with a reference sequence thought to be in the sample, the method comprising the steps of:
copying said RNA to form a cDNA;
polyadenylating the cDNA;
hybridizing the polyadenylated cDNA to a primer bound to a surface;
conducting sequencing by synthesis;
aligning the RNA to a reference sequence;
collecting RNA that does not align to said reference sequence; and
determining the origin of the unaligned RNA.

11. The method of claim 10, further comprising obtaining a RNA sample from a tissue or body fluid of a subject.

12. The method of claim 11, wherein the subject is a human.

13. The method of claim 10, wherein the RNA transcripts are from a human gene.

14. The method of claim 13, wherein the surface comprises a glass surface.

15. The method of any of claim 10 to claim 14, wherein the RNA or cDNA is from about 20 nt to about 500K nt.

16. The method of claim 15, wherein the RNA or cDNA is from about 100 nt to about 100K nt.


SEQUENCE LISTING (70 sequences)

SEQ ID NO: 1 - DNA, 10 bp, Artificial Sequence
  Synthetic oligonucleotide
aaaaaaaaaa

SEQ ID NO: 2 - DNA, 10 bp, Saccharomyces cerevisiae
tttttttaca

SEQ ID NO: 3 - DNA, 127 bp, Saccharomyces cerevisiae
tcaaaagtggttctaccactgatgatgacaaaggtagcgacaaagaggacgttatgggtgatggtagtaacgatgatgacgaagataatgtagacccgctgcaccgtgctaaacaatccagtaacaa

SEQ ID NO: 4 - DNA, 127 bp, Artificial
  Synthetic consensus sequence
tcaaaactggttctaccactgatgatgacaaaggtagcgacaacgaggacgttatgagtgatggtagtaacgatgatgacgaagataatgtagacccgctgcatcgtgctaaacaatccagtaacaa

SEQ ID NO: 5 - DNA, 28 bp, Saccharomyces cerevisiae
caaaactggttctaccactgatgatgac

SEQ ID NO: 6 - DNA, 44 bp, Saccharomyces cerevisiae
gcgacaaagaggacgtttgagtgatggtagtaacgatgatgaca

SEQ ID NO: 7 - DNA, 32 bp, Saccharomyces cerevisiae
agacccgctgcatcgtgctaaacaatccagta

SEQ ID NO: 8 - DNA, 29 bp, Saccharomyces cerevisiae
tcaaaactgttctaccactgatgatgaca

SEQ ID NO: 9 - DNA, 28 bp, Saccharomyces cerevisiae
caaagaggacgttatgagtgatggtagt

SEQ ID NO: 10 - DNA, 25 bp, Saccharomyces cerevisiae
cgaagataatgtagacccgctgcat

SEQ ID NO: 11 - DNA, 18 bp, Saccharomyces cerevisiae
ctaaacatccagtaacaa

SEQ ID NO: 12 - DNA, 39 bp, Saccharomyces cerevisiae
tcaaaactggttctaccactgatgatgacaaaggtagcg

SEQ ID NO: 13 - DNA, 36 bp, Saccharomyces cerevisiae
gaggacgttatgagtgatggtagtcgatgatgacga

SEQ ID NO: 14 - DNA, 36 bp, Saccharomyces cerevisiae
agacccgctgcatcgtgctaaacaatccagtaacaa

SEQ ID NO: 15 - DNA, 32 bp, Saccharomyces cerevisiae
caaaactggttctaccactgatgatgacaaag

SEQ ID NO: 16 - DNA, 42 bp, Saccharomyces cerevisiae
aaagaggacgtatgaggatggtagtaacgatgatgacgaaga

SEQ ID NO: 17 - DNA, 27 bp, Saccharomyces cerevisiae
gacccgctgcatcgtgctaaacaatca

SEQ ID NO: 18 - DNA, 36 bp, Saccharomyces cerevisiae
aaaactggttctaccactgatgatgacaaagtagcg

SEQ ID NO: 19 - DNA, 38 bp, Saccharomyces cerevisiae
ggacgttatgagtgatggtagtaacgatgatgacgaag

SEQ ID NO: 20 - DNA, 30 bp, Saccharomyces cerevisiae
ccgctgccgtgctaaacaatccagtaacaa

SEQ ID NO: 21 - DNA, 37 bp, Saccharomyces cerevisiae
aaaactggttctaccactgatgatgacaaaggtagcg

SEQ ID NO: 22 - DNA, 25 bp, Saccharomyces cerevisiae
ggacgttatgagtgatggtagtaac

SEQ ID NO: 23 - DNA, 40 bp, Saccharomyces cerevisiae
atgacgaagataatgtagacccgctgcatcgtgctaaaca

SEQ ID NO: 24 - DNA, 44 bp, Saccharomyces cerevisiae
aaaactggttctaccactgatgatgacaaagttagcgacaaaga

SEQ ID NO: 25 - DNA, 34 bp, Saccharomyces cerevisiae
cgttatgagtgatggtagtacgatgatgacgaag

SEQ ID NO: 26 - DNA, 29 bp, Saccharomyces cerevisiae
cgctgcatcggctaaacaatccagtaaca

SEQ ID NO: 27 - DNA, 25 bp, Saccharomyces cerevisiae
aactggttctaccactgatgatgac

SEQ ID NO: 28 - DNA, 37 bp, Saccharomyces cerevisiae
cgttatgagtgatgtagtaacgatgatgacgaagata

SEQ ID NO: 29 - DNA, 31 bp, Saccharomyces cerevisiae
cgctgcatcgtgctaaacaatccagtaacaa

SEQ ID NO: 30 - DNA, 35 bp, Saccharomyces cerevisiae
gttctaccactgatatgacaaagtagcgacaaaga

SEQ ID NO: 31 - DNA, 25 bp, Saccharomyces cerevisiae
cgttatgagtgatggtagtaacgat

SEQ ID NO: 32 - DNA, 27 bp, Saccharomyces cerevisiae
gacgaagataatgtagcccgctgctcg

SEQ ID NO: 33 - DNA, 17 bp, Saccharomyces cerevisiae
aaacaatccagtaacaa

SEQ ID NO: 34 - DNA, 27 bp, Saccharomyces cerevisiae
tggttctaccactgatgatgacaaagg

SEQ ID NO: 35 - DNA, 38 bp, Saccharomyces cerevisiae
cgttatgagtgatggtagtaacgatgatgacgaagata

SEQ ID NO: 36 - DNA, 27 bp, Saccharomyces cerevisiae
gcatcgtgctaaacaatccagtaacaa

SEQ ID NO: 37 - DNA, 36 bp, Saccharomyces cerevisiae
ctaccactgatgatgacaaaggtagcgacaaagagg

SEQ ID NO: 38 - DNA, 39 bp, Saccharomyces cerevisiae
atgagtgatggtagtaacgatgatgacgaagataatgta

SEQ ID NO: 39 - DNA, 27 bp, Saccharomyces cerevisiae
gcatcgtgctaaacaatccagtaacaa

SEQ ID NO: 40 - DNA, 51 bp, Saccharomyces cerevisiae
ctaccactgatgatgacaaaggtagcgacaaagaggacgttatgagtgatg

SEQ ID NO: 41 - DNA, 40 bp, Saccharomyces cerevisiae
gtaacgatgatgacgaagataatgtagacccgctgcatct

SEQ ID NO: 42 - DNA, 16 bp, Saccharomyces cerevisiae
aacaatccagtaacaa

SEQ ID NO: 43 - DNA, 36 bp, Saccharomyces cerevisiae; identical to SEQ ID NO: 37

SEQ ID NO: 44 - DNA, 27 bp, Saccharomyces cerevisiae
atgagtgatggtagtaacgatgatgac

SEQ ID NO: 45 - DNA, 38 bp, Saccharomyces cerevisiae
gataatgtagacccgctgcatcgtgctaaacaatccag

SEQ ID NO: 46 - DNA, 47 bp, Saccharomyces cerevisiae
accactgatgatgacaaaggtagcgacaaagggacgttatagtgatg

SEQ ID NO: 47 - DNA, 27 bp, Saccharomyces cerevisiae
aacgatgatgacgaagataatgtagac

SEQ ID NO: 48 - DNA, 26 bp, Saccharomyces cerevisiae
catcgtgctaaacaatccagtaacaa

SEQ ID NO: 49 - DNA, 41 bp, Saccharomyces cerevisiae
atagtcttaagtaatcattcaaaatgccaaagaagagagct

SEQ ID NO: 50 - DNA, 42 bp, Saccharomyces cerevisiae
gatagtcttaagtaatcattcaaaatgccaaagaagagagct

SEQ ID NO: 51 - DNA, 42 bp, Saccharomyces cerevisiae
gatagtctgaagtaatcattcaaaatgccaaagaagagagct

SEQ ID NO: 52 - DNA, 42 bp, Saccharomyces cerevisiae
gatagtcttaagtaatcattcaaaaatgccaaagaaagagct

SEQ ID NO: 53 - DNA, 33 bp, Saccharomyces cerevisiae
atagtcttaagctaatcattcaaaatgccaaag

SEQ ID NO: 54 - DNA, 36 bp, Saccharomyces cerevisiae
atagtcttaagtaatcttcaaaatgcaaagagagag

SEQ ID NO: 55 - DNA, 37 bp, Saccharomyces cerevisiae
atagtcttaagtatatcattcaaaatgcaaagaagag

SEQ ID NO: 56 - DNA, 34 bp, Saccharomyces cerevisiae
atagtcttaagtaacattcaaaatgccaaagaag

SEQ ID NO: 57 - DNA, 30 bp, Saccharomyces cerevisiae
atagtcttagtaatcattcaaaatgccaag

SEQ ID NO: 58 - DNA, 31 bp, Saccharomyces cerevisiae
atagtcttaagtaatctttcaaaatgccaaa

SEQ ID NO: 59 - DNA, 31 bp, Saccharomyces cerevisiae
atagtcttaagtaatattcaaaatgcagaag

SEQ ID NO: 60 - DNA, 33 bp, Saccharomyces cerevisiae
atagtcttaagctaatcttcaaaatgccaaaga

SEQ ID NO: 61 - DNA, 35 bp, Saccharomyces cerevisiae
gagtcttaagtaatcattcaaaatgccaagaagag

SEQ ID NO: 62 - DNA, 34 bp, Saccharomyces cerevisiae
agtcttaagtaatcattcaaaatgccaagaagag

SEQ ID NO: 63 - DNA, 47 bp, Saccharomyces cerevisiae
acaacgtggtagttggcagaatatatatattcttggcaacctagttg

SEQ ID NO: 64 - DNA, 47 bp, Artificial; identical to SEQ ID NO: 63
  Synthetic consensus sequence

SEQ ID NO: 65 - DNA, 31 bp, Saccharomyces cerevisiae
caacgtggtagttggcagaatataatatatt

SEQ ID NO: 66 - DNA, 36 bp, Saccharomyces cerevisiae
acgtggtagttggcagaatatatatattcttggcaa

SEQ ID NO: 67 - DNA, 38 bp, Saccharomyces cerevisiae
acaacgtggtagttggcagaatatatatattctggcaa

SEQ ID NO: 68 - DNA, 33 bp, Saccharomyces cerevisiae
acaacgtggtagttggcagaatatatatattct

SEQ ID NO: 69 - DNA, 45 bp, Saccharomyces cerevisiae
caacgtggtagttggcagaatatatatattcttggcacctagttg

SEQ ID NO: 70 - DNA, 10 bp, Artificial
  Synthetic oligonucleotide
tgtaaaaaat